SEQUENCE LISTING (8 sequences)

SEQ ID NO: 1 - protein, 99 aa, Myxococcus xanthus
  SITE (1)..(93): PrS tag, M1-Y93, 93 amino acid residues
MANITVFYNEDFQGKQVDLPPGNYTRAQLAALGIENNTISSVKVPPGVKAILYQNDGFAGDQIEVVANAEELGPLNNNVSSIRVISVPVQPRYLVPRGS

SEQ ID NO: 2 - protein, 191 aa, Myxococcus xanthus
  SITE (1)..(185): PrS2 tag M1-Y185, 185 amino acid residues
MANITVFYNEDFQGKQVDLPPGNYTRAQLAALGIENNTISSVKVPPGVKAILYQNDGFAGDQIEVVANAEELGPLNNNVSSIRVISVPVQPRMANITVFYNEDFQGKQVDLPPGNYTRAQLAALGIENNTISSVKVPPGVKAILYQNDGFAGDQIEVVANAEELGPLNNNVSSIRVISVPVQPRYLVPRGS

SEQ ID NO: 3 - DNA, 114 bp, Artificial Sequence
  DNA sequence of TEV cleavage site, multiple cloning site, thrombin cleavage site, and His6 tag designed for pCold(PST) vector
cggggtaccgaaaacctgtattttcagggagctgcagctcatatggtcgacctcgagggatccctggtgccacgcggtagtcatcaccatcaccatcactaatctagaattatt

SEQ ID NO: 4 - DNA, 66 bp, Artificial Sequence
  DNA sequence of TEV cleavage site, multiple cloning site designed for T-REx(PST)
ggtaccgaaaacctgtattttcagggagctgcagctcatatggtcgacctcgagggatcctctaga

SEQ ID NO: 5 - DNA, 60 bp, Artificial Sequence
  DNA sequence of multiple cloning site and thrombin cleavage site
cccaggtacctggtgccacgcggtagtcatatggtcgacctcgagggatcctaatctaga

SEQ ID NO: 6 - protein, 10 aa, Unknown
  c-myc epitope
EQKLISEEDL

SEQ ID NO: 7 - DNA, 18 bp, Artificial Sequence
  Thrombin cleavage site
ctggtgccacgcggtagt

SEQ ID NO: 8 - protein, 6 aa, Artificial Sequence
  Thrombin cleavage site
LVPRGS

(19) United States
(12) Patent Application Publication
(10) Pub. No.: US 2009/0215120 A1
(43) Pub. Date: Aug. 27, 2009

(54) The Use of Protein S Fusion for Protein Solubilization

(75) Inventors: Masayori Inouye, New Brunswick, NJ (US); Takeshi Yoshida, Rochester, NY (US)

Correspondence Address: PATENT DOCKET ADMINISTRATOR;LOWENSTEIN SANDLER PC, 65 LIVINGSTON AVENUE, ROSELAND, NJ, 07068, US

(73) Assignee: UNIVERSITY OF MEDICINE AND DENTISTRY OF NEW JERSEY, Somerset, NJ (US)

(21) Appl. No.: 12/293,866

(22) PCT Filed: Mar. 20, 2007
(86) PCT No.: PCT/US2007/064457

§ 371 Date: Apr. 20, 2009

Related U.S. Application Data
(60) Provisional application No. 60/783,998, filed on Mar. 20, 2006.

(51) Int. Cl. C12P 21/00; C12N 15/00; C07K 14/00; C07K 1/00; G01N 33/68

(52) U.S. Cl. 435/69.7; 435/320.1; 530/350; 530/413; 436/86

(57) ABSTRACT

The invention provides vectors containing a multiple cloning site comprising a PrS tag or a PrS2 tag from Myxococcus xanthus. Methods are provided for enhancing solubility of a target protein using Protein S tagged target fusion proteins.

### CROSS-REFERENCE TO RELATED APPLICATION

This application claims priority to U.S. Provisional Application No. 60/783,998entitled “The Use of Protein S Fusion for Protein Solubilization” by Inouye et al., filed on Mar. 20, 2006. The entire disclosure of this application is incorporated herein by reference.

### FIELD OF THE INVENTION

The present invention relates to systems for enhancing the production and solubility of proteins.

### STATEMENT UNDER C.F.R. 1.821(f)

In accordance with § 1.821(f), the content of the attached paper copy and the attached computer readable copy “18622-12 umdnj.st25.txt” of the Sequence Listing submitted in accordance with 37 C.F.R. § 1.821 (c) and (e), respectively, are identical.

### BACKGROUND OF THE INVENTION

Protein expression is a major challenge in medical science and biotechnology, as a large number of proteins become insoluble when they are expressed in various conventional expression systems. A number of approaches have been developed for the isolation and purification of proteins, particularly recombinant proteins, from other components of a biological sample. These purification methods include, inter alia, ion exchange chromatography, gel filtration, and affinity chromatography. Affinity chromatography is more efficient than the other methods, because of the specificity of the target protein or of a target protein's tag for a purifying reagent, such as ah antibody or ligand, to which it binds.

Tagging of a protein for its affinity purification has been a method of choice for protein purification for many years and currently is in use in proteomic studies in yeast and to a lesser extent in higher eukaryotes. A large number of unique tags from small peptides as short as six amino acid residues (His tag) to a large protein as much as 40 kDa (MBP) are available (see review: Stevens, R. C., 2000. Structure Fold Des 8:R177-85). Among the tags used, His-tag has been the most widely and successfully used, as His-tagged proteins can be specifically trapped on Ni-NTA (nickel-nitrilotriacetic acid) resin, which can be eluted by EDTA or imidazol. Other protein tags such as maltose-binding protein (MBP), Staphylococcal protein A, calmodulin-binding peptide (CBP) and glutathione-S-transferase (GST) have also been used for both prokaryotic and eukaryotic proteins. However, many of these affinity technologies are not specific enough to purify the lagged proteins in one step, as non-specific proteins are also frequently trapped by the resin used. To overcome this problem, an affinity purification system using two different tags tandemly fused to the N-terminal end of a protein has been developed (Rigaut, G., et al. 1999. Nat Biotechnol 17:1030-2). This TAP tag (Tandem Affinity Purification) technology has been shown to be very effective in identifying protein factors that form complexes with a protein of interest.

Disadvantages of current fusion tagging systems include low solubility of tagged fusion proteins and inability to purify tagged proteins under conditions that are common in many protein purification protocols. Furthermore, current fusion tags are difficult for use for analytic studes of protein structure. For example, the His tag cannot be used for experiments designed for NMR structural studies because the Ni-NTA resin cannot be used for NMR spectroscopy as paramagnetic effect of Ni⁺⁺ ion leads to broadening of peaks and interferes with data collection.

Thus, there is a need for an improved tagging system that allows tagged fusion proteins to remain soluble and stable for purification and study of proteins, including analytical techniques such as protein purification, NMR and x-ray crystallography, and identification of protein factors interacting with a specific protein. The Protein S Tag technology of the present invention is shown herein to be an excellent fusion tag for improving expression of unstable proteins.

Protein S is a spore coat protein of M. xanthus, a developmental gram-negative bacterium that forms multi-cellular fruiting bodies upon nutritional depletion (see (Dworkin, M., et al. 1985. Science 230:18-24, Shimkets, L. J. et al. 1990. Microbiol Rev 54:473-501) for a review). In fruiting bodies, cells are converted into uniform spherical spores (myxospores) of the diameter of 1 μm, which are highly resistant to dessication and heat. It has been shown that myxospores have a major coat protein termed protein S that assembles on the surface of the myxospore in a Ca⁺⁺-dependent manner (Inouye, M., et al., 1979. Proc Natl Acad Sci USA 76:209-13).

Protein S from Myxococcus xanthus is unique in that it significantly enhances the production and solubility of a protein when it is fused to the N-terminus of that protein. Importantly, results of experiments related to this invention show that fusion of protein S with OmpR (a transcription factor) does not affect the properties of OmpR (Harlocker, S. L., et al. 1995. J Biol Chem 270:26849-56), indicating that the protein S domain is folded independently and will not interact target proteins of interest. We have now found that protein S tagged proteins can be specifically trapped on myxospores, which are easily purified from fruiting bodies of M. xanthus.

There are several unique features of protein S. Protein S can be readily released in the presence of EDTA or at high salt concentrations. Furthermore, purified protein S can be easily reassembled on the surface of myxospores upon addition of Ca⁺⁺ (Inouye, M., et al., 1979. Proc Natl Acad Sci USA 76:209-13). A single myxospore can bind up to 3.3×10⁶ protein S molecules assembled on its surface (Inouye, M., et al., 1979. Proc Natl Acad Sci USA 76:209-13). Its NMR solution structure has been determined (FIG. 1A; (Bagby, S., et al. 1994. Structure 2:107-22), which consists of 173 amino acid residues and has two tandem Ca⁺⁺-binding domains. Two Ca⁺⁺-binding domains are superimposable and consist of 7 β-strands and one α helix (FIG. 1A). Protein S crystals have been shown to be highly resistant to X-ray radiation (Inouye, S., 1980. J Biol Chem 255:3713-4). These advantages of protein S are useful for a wide variety of applications, as shown in this invention.

### SUMMARY OF THE INVENTION

The present invention provides a vector containing a multiple cloning site comprising a PrS tag or a PrS2 tag from Myxococcus xanthus.

The present invention also provides a method for enhancing solubility of a target protein, comprising: fusing a nucleic acid sequence encoding at least one N-terminal domain of Protein S (PrS tag) from Myxococcus xanthus to a nucleic acid sequence encoding the target protein to create a nucleic acid sequence encoding a PrS tagged target protein; positioning the Protein S tagged target protein of step (a) into a vector; transforming a host cell with the vector; and culturing the host cell under conditions suitable for gene expression, whereby the expressed PrS tagged target protein is more soluble than the untagged target protein. In one embodiment, a tandemly repeating N-terminal domain of PrS is used, called a PrS₂ tag.

The present invention further provides a PrS-tagged protein molecule bound to one or more myxospores of Myxococcus xanthus.

Also provided is a method for purifying a PrS-tagged protein comprising contacting the PrS-tagged protein with an affinity resin comprising myxospores of Myxococcus xanthus, thereby immobilizing the Protein S tagged protein on the affinity resin.

Also provided is a method of preparing a target protein for an analytical study, comprising preparing a PrS-tagged target protein, and performing the analytical study.

The present invention additionally provides a method of characterizing a target protein comprising fusing at least one PrS tag or a PrS₂ tag to the target protein, performing nuclear magnetic resonance spectroscopy and analyzing data from the nuclear magnetic resonance spectroscopy.

### BRIEF DESCRIPTION OF THE FIGURES

FIG. 1. The NMR Structure of Protein S and Scheme of PrS₂ Tagged Protein. A. NMR structure of M. xanthus protein S with Ca⁺⁺ ions bound (Bagby, S., et al. 1994. Structure 2:107-22). B. N-terminal domain (1-92) of protein S used for PrS₂-tag is shown. Two tandem repeats of protein S(1-92) (PrS₂) (oval) is fused at N-terminal end of a protein of interest (white box).

FIG. 2. Comparison of Wild-Type OmpR and PrS₂-OmpR. A. The complex formation between EnvZc and PrS₂-OmpR. EnvZc (4 μM) and PrS₂-OmpR (4 μM) (lane 4) or OmpR (4 μM) (lane 2) were mixed and incubated in the reaction buffer at room temperature for 5 min. The samples were subjected to 10% native PAGE. B. Phosphotransfer from phosphorylated EnvZc (EnvZc-P) to PrS₂-OmpR. Purified ³²P-labelled EnvZc-P (2 μM) was mixed with OmpR (4 μM), PrS₂-OmpR (4 μM), or the mixture of OmpR (2 μM) and PrS₂-OmpR (2 μM) in the reaction buffer [50 mM Tris-HCl (pH 8.0), 50 mM KCl, 5 mM CaCl₂, 5% glycerol]. The final reaction mixtures were incubated at room temperature. Aliquots were removed at 20, 40, 60, and 120 sec and the reaction was stopped with 5×SDS loading buffer. The samples were subjected to 17.5% SDS-PAGE. C. Dephosphorylation of phosphorylated PrS₂-OmpR (PrS₂-OmpR-P) by EnvZc. First purified ³²P-labelled EnvZc-P (2 μM) was mixed with OmpR (4 μM), PrS₂-OmpR (4 μM), or the mixture of OmpR (2 μM) and PrS₂-OmpR (2 μM) and incubated in the reaction buffer [50 mM Tri-HCl (pH 8.0), 50 mM KCl, 5 mM CaCl₂, 5% glycerol] at room temperature for 2 min to generate OmpR-P or PrS₂-OmpR-P. After adding ADP at the final concentration of 1 mM, aliquots were removed at 20, 40, 60, and 120 sec and the reaction was stopped with 5×SDS loading buffer. The samples were subjected to 17.5% SDS-PAGE. D. DMA binding of PrS₂-OmpR-P. Various mixtures of OmpR-P and PrS₂-OmpR-P as indicated on the top of the gel were mixed with 30-bp F1F2a DNA fragment in the DNA binding buffer. DNA gel mobility-shift assay was carried out with the 5′-end labeled DNA fragment, a; two OmpR-P molecules/F1F2a complex, b; one OmpR-P and one PrS₂-OmpR-P molecules/F1F2a complex, and c; two PrS₂-OmpR-P molecules/F1F2a complex. The gels from B, C, and D were dried and exposed for autoradiography.

FIG. 3. Characterization of Binding of PrS₂-OmpR to Myxospores. A. Phase light microscope picture of myxospores (100× magnification). B. PrS₂-OmpR binds to myxospores in a Ca⁺⁺-dependent manner. Purified PrS₂-OmpR (3 μg) was mixed with myxospores and incubated in 50 mM Tris-HCl buffer containing 50 mM KCl in the presence of 20 mM EDTA, 10 mM MgCl₂, or 10 mM CaCl₂ at 4° C. for 1 hr. After washing, myxospores were collected by centrifugation and suspended in SDS loading buffer. The samples were incubated in a boiling-water bath for 5 min and their supernatant were subjected to 17.5% SDS-PAGE. Lane 1, the total amount of purified PrS₂-OmpR used. C. EDTA can release PrS₂-OmpR bound to myxospores. First, PrS₂-OmpR binding to myxospores was carried out in the presence of CaCl₂. Myxospores were collected by centrifugation and washed with the same buffer as used for binding. The collected myxospores were incubated in 50 mM Tris-HCl buffer containing 50 mM KCl in the presence of 20 mM EDTA or 10 mM CaCl₂ for 15 min. Lane 1, the total amount of PrS₂-OmpR used, and lane 6, the total PrS₂-OmpR bound to myxospores. D. Purified PrS₂-OmpR added to an E. coli lysate is specifically trapped by myxospores. Purified PrS₂-OmpR (3 μg) was mixed with an E. coli BL21(DE3) lysate and specific binding of PrS₂-OmpR to myxospores was examined (lane 5). Lane 1, the total purified PrS₂-OmpR used; lane 2, purified PrS₂-OmpR was added without cell lysate; lane 3, cell lysate alone was used; lane 4, cell lysate was added without PrS₂-OmpR. E. PrS₂-OmpR can bind to myxospores even in the presence of 4 M urea. PrS₂-OmpR binding was examined in the presence of urea (2, 3, and 4 M). Lanes 3-5, without the cell lysate; lanes 7-9, with the cell lysate; lane 6, the mixture of PrS₂-OmpR and E. coli BL21(DE3) lysate used.

FIG. 4. DNA Pull-Down Experiment. A. Purified EnvZc11 (used to phosphorylate OmpR) and DNA fragment of the upstream region of the ompF promoter (ompF^{P}) was mixed and the mixture was incubated in 50 mM Tris-HCl (pH 8.0) buffer, containing 50 mM KCl, 10 mM CaCl₂, and 1 mM ATP, in the absence (lane 3) or presence of PrS₂-OmpR (lane 4) for 30 min at room temperature. Myxospores were added to the mixture, which was further incubated for 1 hr at 4° C. Myxospores were collected by centrifugation and the supernatant of the samples were subjected to 1.7% agarose gel electrophoresis. Lane 1, 100-bp marker (Bio-Rad); lane 2, ompF^{P} DNA fragment. B. The same experiment as described above was carried out using a mixture of two different linearized plasmids; pCR-ompF^{P} digested by BamH I and pET-EnvZc digested by EcoR I. The former plasmid contains an ompF^{P} DNA fragment used in A above, while the latter plasmid contains no OmpR binding sites. The samples were subjected to 0.8% agarose gel electrophoresis. Lane 1, pCR-ompF^{P} DNA fragment and pET-EnvZc DNA fragment; lane 4, λ/Hind III marker.

FIG. 5. A Plasmid Map of pCold(PST) Vector Derived From pCold(PST)III. A. pCold(PST) is derived from pColdIII vector (Qing, G., et al. 2004. Nat Biotechnol 22:877-82). TEE: Translation Enhancing Element. B. DNA sequence of TEV cleavage site, multiple cloning site, thrombin cleavage site, and His₆ tag designed for pCold(PST) vector.

FIG. 6. DNA Sequence of TEV Cleavage Site, Multiple Cloning Site Designed for T-REx(PST). c-myc epitope: [Glu-Gln-Lys-Leu-Ile-Ser-Glu-Glu-Asp-Leu]

FIG. 7. A Plasmid Map of pCold(PST) Vector Derived From pCold(PST)IV (a) The scheme of an expression vector, pCold(PST). pCold(PST) is constructed using pCold IV, which is one of the cold-shock vectors and is available from Takara Bio Inc., Shiga, Japan. PrS tag is showing where DNA fragment of either PrS tag or PrS₂ tag is inserted. PrS tag consists of the N-terminal domain of protein S from Met1-Arg82 plus Tyr93 while PrS₂ tag consists of two tandem repeated sequence of PrS tag(Met1-Arg82) plus Tyr185. (b) The sequence of Multiple cloning site and thrombin cleavage site. Thrombin cleavage site, LVPRGS; CTG GTG CCA CGC GGT AGT, is introduced between Tyr83 of PrS tag or Tyr185 of PrS₂ tag and multiple cloning site containing NdeI, SalI, XhoI, BamHI, and XbaI sites. cspA 3′-UTR, Translation Enhance Element, cspA 5′-UTR, lac operator, and cspA promoter are from the original pCold IV vector.

### DETAILED DESCRIPTION OF THE INVENTION

The invention provides a novel protein technology termed Protein S Tag (PST). Also provided is a novel approach for the structural study of proteins, whose structures cannot be determined by conventional methods.

We can apply the technology of the invention for characterization of proteins, either from prokaryotes or from eukaryotes, including human proteins. Since the expression system is designed in such a way that PrS or PrS₂ tag can be cleaved off by a specific protease such as TEV protease or thrombin, one can get soluble proteins identical to their natural forms. Importantly, we demonstrated that both PrS and PrS₂ tag have almost no effect on the function of a target protein (Harlocker S L et al. J Biol Chem. 1995). If a target protein becomes insoluble after cleaving PrS or PrS₂ tag, one can still characterize the function of a target protein as a fusion protein with PrS or PrS₂ tag.

When proteins are expressed as fusion proteins with the N-terminal domain of protein S of Myxococcus xanthus, they become expressed as soluble proteins. Results from our studies show that the solubility and expression levels of a number of insoluble or poor expression of eukaryotic proteins from Human, Drosophila, Caenorhabditis elegans, as well as proteins from Escherichia coli are significantly improved when they are expressed as fusion proteins with protein S. Otherwise, proteins are expressed in insoluble forms. For these as well as for other applications, one single N-terminal domain of protein S termed “PrS tag” and two tandem-repeated N-terminal domain of protein S from Myxococcus xanthus termed “PrS₂ tag” influence target proteins differently. Therefore, expression systems to express a target protein with either the PrS tag or the PrS₂ tag are provided herein. The invention also provides expression systems with more than two N-terminal domains of protein S from M. xanthus.

In a “chimeric molecule,” two or more molecules that are capable of existing separately are joined together to form a single molecule having the desired functionality of all of its constituent molecules. The constituent molecules of a chimeric molecule can be joined synthetically by chemical conjugation or, where the constituent molecules are all polypeptides, polynucleotides encoding the polypeptides may be fused together recombinantly such that a single continuous polypeptide is expressed. Such a chimeric polypeptide is termed a “fusion protein.” A “fusion protein” is a chimeric molecule in which the constituent molecules are all polypeptides and are attached (fused) to each other such that the chimeric molecule forms a continuous single chain. The various constituents can be directly attached to each other or can be coupled through one or more peptide linkers.

A “linker” as used in reference to a chimeric molecule, such as the fusion proteins of the invention, refers to any molecule that links or joins the constituent molecules of the chimeric molecule. Where the chimeric molecule is a fusion protein, the linker may be a peptide that joins the proteins comprising a fusion protein. Although a linker generally has no specific biological activity other than to join the proteins or to preserve some minimum distance or other spatial relationship between them, the constituent amino acids of a peptide spacer may be selected to influence some property of the molecule such as the folding, net charge, or hydrophobicity. A peptide linker may optionally include a site for digestion by a protease, for separation of the fused constituent polypeptides. For preparing proteins for Nuclear Magnetic Resonance studies, discussed below, preferred linkers comprise about 1 to about 20 amino acid residues. Particularly preferred linkers comprise about 3 to about 10amino acid residues.

Construction of Protein S Tag (PST) Expression System in E. coli and Mammalian Cells

As used herein, the terms “vector” and “expression vector” refer to a replicon, i.e., any agent that acts as a carrier or transporter, such as a bacteriophage (phage), plasmid, phagemid, cosmid, bacmid or virus, to which another genetic sequence or element (either DNA or RNA) may be attached so as to bring about the replication of the attached sequence or element and so that sequence or element can be conveyed into a host cell. The E. coli vector system described herein utilizes pCold vectors (available from Takara Bio Inc., Shiga, Japan), which induce protein production at low temperatures.

The invention provides an E. coli expression system using pCold cold-shock vectors for protein purification and NMR structural studies. A mammalian tetracycline-inducible system can be used for identification of protein factors that form a complex with a protein of interest in living cells. This protein of interest is called a “target protein” herein, which refers to a protein, polypeptide or fragments thereof to which a PrS or PrS₂ tag is attached. In preferred embodiments, the target molecule is a protein.

The invention provides a vector containing a multiple cloning site comprising a nucleic acid sequence encoding a PrS tag (SEQ ID NO: 1) or a PrS₂ tag (SEQ ID NO: 2) from Myxococcus xanthus. In other embodiments, the invention uses one or more tags obtained from the N-terminal domain of Protein S from Myxococcus xanthus. The PrS and PrS₂ tags are preferably amino acid residues 1-93 of Protein S (SEQ ID NO: 1 and SEQ ID NO: 2), but may also be 1-92 residues or 1-94 residues, or may be any amino acid residues between 1 and 173 of Protein S.

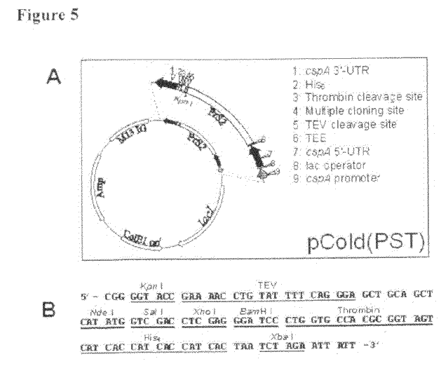

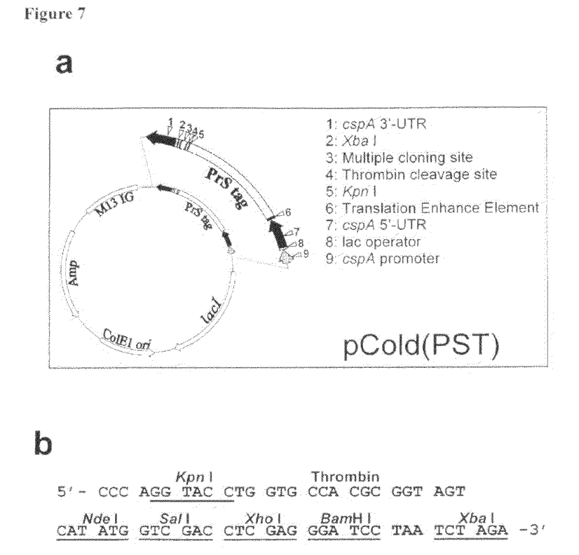

In preferred embodiments, the multiple cloning site of the vector of the invention comprises SEQ ID NO: 1 or SEQ ID NO: 2. The vector may be a high expression cold shock vector such as a pColdIII or pColdIV vector. The pColdIII vector is preferable for expression in E. coli since the Translation Enhancing Element (TEE) of the vector (see FIG. 5) provides better expression. The pColdIV expression without TEE (FIG. 7) is useful in cases where TEE has a negative effect on translation.

In a mammalian system, the preferred vector is a tetracycline-inducible PST expression system from a Human Embryonic Kidney 293 cell line (TREx-PST) harboring PrS₂-tagged protein. Thus, the invention provides vectors expressed in mammalian host cell, including human cells.

As used herein, the terms “encode”, “encoding” or “encoded”, with respect to a specified nucleic acid, refers to information stored in a nucleic acid for translation into a specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code.

The term “codon” as used herein refers to triplets of nucleotides that together specify an amino acid residue in a polypeptide chain. Most organisms use 20 or 21 amino acids to make their polypeptides, which arc proteins or protein precursors. Because there are four possible nucleotides, adenine (A), guanine (G), cytosine (C) and thymine (T) in DNA, there are 64 possible triplets to recognize only 20 amino acids plus the termination signal. Due to this redundancy, most amino acids are coded by more than one triplet. The codons that specify a single amino acid are not used with equal frequency. Different organisms often show particular “preferences” for one of the several codons that encode the same given amino acids. If the coding region contains a high level or a cluster of rare codons, removal of the rare codons by resynthesis of the gene or by mutagenesis can increase expression. See J. Sambrook and D. W. Russell, Molecular Cloning: A Laboratory Manual, Third Edition, Cold Spring Harbor Press, Cold Spring Harbor, N.Y. (2001), at 15.12; which is incorporated herein v by reference. “Codon selection” therefore may be made to optimize expression in a selected host. The most preferred codons are those which are frequently found in highly expressed genes. For “codon preferences” in E. coli, see Konigsberg, et al., Proc. Nat'l. Acad. Sci. U.S.A. 80:687-91 (1983), which is incorporated herein by reference.

One of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. The term “conservatively modified variants” applies to both amino acid and nucleic acid sequences. With respect to particular nucleic acid sequences, conservatively modified variants refers to those nucleic acids which encode identical or conservatively modified variants of the amino acid sequences. Because of the degeneracy of the genetic code, a large number of functionally identical nucleic acids encode any given protein. For instance, the codons UUA, UUG, CUU, CUC, CUA, and CUG all encode the amino acid leucine. Thus, at every position where a leucine is specified by a codon, the codon can be altered to any of the corresponding codons described without altering the encoded polypeptide. Such nucleic acid variations are “silent variations” and represent one species of conservatively modified variation. Every nucleic acid sequence herein which encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is within the scope of the present invention.

The present invention includes active portions, fragments, derivatives, mutants, and functional variants of protein S that retain any of the biological properties of protein S.

One member of a binding pair may be used to “tag” a protein of interest, with the other member used as an affinity ligand or “affinity resin”. Such a protein “tag” may be “fused” recombinantly and expressed to produce a fusion protein with the tag attached. The “tagged” fusion protein is then affinity purified by interaction with the binding partner of the tag and the tag is then optionally cleaved to release pure protein.

The term “gene” refers to an ordered sequence of nucleotides located in a particular position on a segment of DNA that encodes a specific functional product (i.e, a protein or RNA molecule). It can include regions preceding and following the coding DNA as well as introns between the exons.

The term “induce” or inducible” refers to a gene or gene product whose transcription or synthesis is increased by exposure of the cells to an inducer or to a condition, e.g., heat.

The terms “inducer” or “inducing agent” refer to a low molecular weight compound or a physical agent that associates with a repressor protein to produce a complex that no longer can bind to the operator.

The term “induction” refers to the act or process of causing some specific effect, for example, the transcription of a specific gene or operon, or the production of a protein by an organism after it is exposed to a specific stimulus.

The terms “introduced,” “transfection,” “transformation” and “transduction” in the context of inserting a nucleic acid into a cell include reference to the incorporation of a nucleic acid into a prokaryotic cell or eukaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The term “isolated” refers to material, such as a nucleic acid or a protein, which is substantially free from components that normally accompany or interact with it as found in its naturally occurring environment. The isolated material optionally comprises material not found with the material in its natural environment; or, if the material is in its natural environment, the material has been synthetically (non-naturally) altered by deliberate human intervention. For example, an “isolated nucleic acid” may comprise a DNA molecule inserted into a vector, such as a plasmid or virus vector, or integrated into the genomic DNA of a prokaryotic or eukaryotic cell or host organism. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it is generally associated in its natural state (i.e., in cells or tissues). An isolated nucleic acid (either DNA or RNA) may further represent a molecule produced directly by biological or synthetic means and separated from other components present during its production.

As used herein, the term “nucleic acid” or “nucleic acid sequence” includes any DNA or RNA molecule, either single or double stranded, and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. Unless otherwise limited, the term encompasses known analogs.

The term “oligonucleotide” refers to a nucleic acid molecule comprised of two or more ribo- or deoxyribonucleotides, preferably more than three, joined by phosphodiester bonds.

The term “operator” refers to the region of DNA that is upstream (5′) from a gene(s) and to which one or more regulatory proteins (repressor or activator) bind to control the expression of the gene(s)

As used herein, the term “operon” refers to a functionally integrated genetic unit for the control of gene expression. It consists of one or more genes that encode one or more polypeptide(s) and the adjacent site (promoter and operator) that controls their expression by regulating the transcription of the structural genes. The term “expression operon” refers to a nucleic acid segment that may possess transcriptional and translational control sequences, such as promoters, enhancers, translational start signals, polyadenylation signals, terminators, and the like, and which facilitate the expression of a polypeptide coding sequence in a host cell or organism.

The phrase “operably linked” includes reference to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

The abbreviation “ORF” stands for “open reading frame, a portion of a gene's sequence that contains a sequence of bases, uninterrupted by internal stop sequences, and which has the potential to encode a peptide or protein. Open reading frames start with a start codon, and end with a termination codon. A termination or stop codon determines the end of a polypeptide.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The abbreviation “PCR” refers to polymerase chain reaction, which is a technique for amplifying the quantity of DNA, thus making the DNA easier to isolate, clone and sequence. See, e.g., U.S. Pat. Nos. 5,656,493, 5,33,675, 5,234,824, and 5,187,083, each of which is incorporated herein by reference.

As used herein the term “promoter” includes reference to a region of DNA upstream (5′) from the start of transcription and involved in recognition and binding of RNA polymerase and other proteins to initiate transcription. The term “inducible promoter” refers to a promoter that may be activated in response to either the presence of a particular compound, i.e., the inducer or inducing agent, or to a defined external condition, e.g., elevated temperature.

The phrase “site-directed mutagenesis” refers to an in vitro technique whereby base changes i.e., mutations, are introduced into a piece of DNA at a specific site, using recombinant DNA methods.

The term “untranslated region” or UTR, as used herein refers to a portion of DNA whose bases are not involved in protein synthesis.

The terms “variants”, “mutants” and “derivatives” of particular sequences of nucleic acids refer to nucleic acid sequences that are closely related to a particular sequence but which may possess, either naturally or by design, changes in sequence or structure. By “closely related”, it is meant that at least about 60%, but often, more than 85%, of the nucleotides of the sequence match over the defined length of the nucleic acid sequence. Changes or differences in nucleotide sequence between closely related nucleic acid sequences may represent nucleotide changes in the sequence that arise during the course of normal replication or duplication in nature of the particular nucleic acid sequence. Other changes may be specifically designed and introduced into the sequence for specific purposes. Such specific changes may be made in vitro using a variety of mutagenesis techniques. Such sequence variants generated specifically may be referred to as “mutants” or “derivatives” of the original sequence.

A skilled artisan likewise can produce protein variants having single or multiple amino acid substitutions, deletions, additions or replacements. These variants may include inter alia: (a) variants in which one or more amino acid residues are substituted with conservative or non-conservative amino acids; (b) variants in which one or more amino acids are added; (c) variants in which at least one amino acid includes a substituent group; (d) variants in which amino acid residues from one species are substituted for the corresponding residue in another species, either at conserved or non-conserved positions; and (d) variants in which a target protein is fused with another peptide or polypeptide such as a fusion partner, a protein tag or other chemical moiety, that may confer useful properties to the target protein, such as, for example, an epitope for an antibody. The techniques for obtaining such variants, including genetic (suppressions, deletions, mutations, etc.), chemical, and enzymatic techniques are known to the skilled artisan.

### Affinity Purification

### The Use of Myxospores for Affinity Purification

The invention also provides a method for purifying a PrS-tagged or PrS₂-tagged protein comprising contacting the tagged protein with an affinity resin comprising myxospores of Myxococcus xanthus, thereby immobilizing the PrS-tagged or PrS₂-tagged protein on the affinity resin. A PrS-tagged or PrS₂-tagged protein molecule bound to one or more myxospores of Myxococcus xanthus is also provided. The PrS or PrS₂ tag can bind to the surface of myxospores (spores of M. xanthus) in the presence of Ca²⁺ or Mg²⁺. In one embodiment of this purification method, the PrS-tagged or PrS₂-lagged protein is contacted with the affinity resin in the presence of Ca²⁺ or Mg²⁺. The PrS-tagged or PrS₂-tagged protein is released from the affinity resin by adding an agent that chelates calcium or magnesium. Thus, using myxospores, a PrS₂-tagged target protein can be specifically isolated as it binds to the surface of a myxospore and easily released from the surface of a myxospore by adding a chelating reagent such as EDTA or EGTA.

For use as a resin in affinity purification of proteins, myxospores are washed with 25 mM EDTA to remove protein S and protein C present on the surface of myxospores. Purified myxospores consist of uniform spherical particles of the diameter of approximately 1 μm. These can be homogeneously suspended in solution for an extended period of time. Notably, myxospores have a high absorbing capacity for protein S (3.3×10⁶ molecules/spore), which is also ideal for affinity purification of proteins.

The affinity purification of this invention is readily carried out by simply incubating myxospores with cell lysates for 1 hr at 4° C., following by collection of these myxospores by low-speed centrifugation. The affinity of fusion proteins to myxospores is in a range of 10⁻⁹ M and myxospores can specifically trap fusion proteins that are expressed at very low levels in E. coli cells.

The invention also provides a protein purified according to the methods of the invention. In a preferred embodiment, the purified protein is a PrS-tagged or PrS₂-tagged protein.

The invention also provides a kit comprising an affinity resin and instructions for use in a method of purifying a PrS-tagged protein. The kit further comprises components for producing a PrS-tagged protein.

The term “affinity ligand” or “affinity reagent” refers to an agent that, specifically binds a cognate ligand with high affinity. Such agents may be attached to a support, termed “substrate,” “resin” or “matrix material,” to form an “affinity matrix” or “resin matrix” or “affinity resin.” An affinity resin of this invention comprises spores of M. xanthus called “myxospores” of Myxococcus xanthus or a derivative or fragment thereof that is capable of binding its respective cognate ligand. A cognate ligand may be “immobilized” or “retained” or “bound” on a a matrix until released with a releasing agent.

A “releasing agent” refers to a composition that is capable of releasing an immobilized, bound molecule from an affinity matrix (e.g. releases a bound PrS- or PrS₂-tagged molecule from a myxospore affinity resin matrix). Releasing agents of this invention can work through a variety of mechanisms including sequestering divalent cations, denaturation of a protein, and protease digestion to separate a bound tag from the molecule to which it is attached.

### Analytical Techniques:

The method of this invention is applicable to characterization of proteins, either from prokaryotes or from eukaryotes, including human proteins. Since the expression system is designed in such a way that PrS or PrS₂ tag can be cleaved off by a specific protease such as TEV protease or thrombin, one can get soluble proteins identical to their natural forms. Previously, we have demonstrated that both PrS and PrS₂ tag have negligible or no effect on the function of a target protein (Harlocker S L et al. J Biol Chem. 1995). Therefore, if a target protein becomes insoluble after cleaving PrS or PrS₂ tag, it is still possible to characterize the function of a target protein as a fusion protein with the PrS or PrS₂ tag.

Importantly, the PrS and PrS₂ tags of the invention are highly stable and resistant to proteases. Furthermore, the structure of protein S has been determined by NMR (Bagby S et al. Proc Natl Acad Sci USA. 1994, Wenk M et al. J Mol Biol. 1999) and has been shown to form a crystal easily. By using these advantages, the PrS and PrS₂ tags of the invention can be applied to structural studies such as nuclear magnetic resonance (NMR) and X-ray crystallography of unstable proteins at high concentrations. We have demonstrated that NMR spectra of a target protein fused with a PrS₂ tag is not affected by the PrS₂ tag. The PrS₂ tag improved the formation of crystal of a target protein when the target protein was fused with PrS₂ tag.

### NMR Structural Study of Protein S-tagged Proteins Trapped on Myxospores

There are a large number of proteins whose three-dimensional structures cannot be determined by the conventional technologies (X-ray and NMR), as these proteins are either unstable or are poorly soluble in solution. The technology provided by the invention overcomes this problem by using PrS- or PrS₂-tagged proteins trapped on myxospores.

Using this property of PrS₂ tag binding to the surface of a myxospore, it is also possible to eliminate the spectra of PrS₂ tag from NMR spectra of a PrS₂-tagged target protein. As interaction of PrS₂ tag to the surface of myxospores limits its movement, it will significantly reduce its signal. Such a limiting effect resulting in a reduced signal is known as a broadening effect. In the other hand, a target protein fused to PrS₂ tag by a flexible linker retains its free rotational movement, resulting in giving satisfactory signals for NMR study. It is important to note that the linker connecting PrS₂ tag with a target protein has a crucial role in cystallization and NMR studies of PrS₂-tagged fusion proteins. Therefore, it is useful to design the expression system with linkers of differing lengths. Preferred linkers comprise about 1 to about 20 amino acid residues. Particularly preferred linkers comprise about 3 to about 10 amino acid residues. A method of claim 1, wherein the PrS-tagged protein comprises a linker between the protein S and the target protein.

Methods of the invention include preparing a target protein for an analytical study, comprising preparing a PrS-tagged or PrS₂-tagged target protein, and performing the analytical study. Other studies useful in this invention besides NMR include drug screening assays, binding assays and X-ray crystallography or any study requiring a soluble target protein.

As used herein and in the appended claims, the singular forms “a”, “and”, and “the” include plural referents unless the context clearly dictates otherwise.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Where a range of values is provided herein, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Protein S-OmpR Fusion

OmpR is a transcription factor of E. coli required for the reciprocal expression of ompF and ompC genes that encode major outer membrane proteins. Previously, it was fused to two tandem repeats of the N-terminal domain (residues 1-92) of protein S (termed PrS₂-OmpR) to determine the exact number of OmpR-binding sites in the upstream region of the ompF promoter (Harlocker, S. L., et al. 1995. J Biol Chem 270:26849-56). This fusion of protein S at the N-terminal end of OmpR did not hamper the DNA binding ability of OmpR, thus allowing formation of a ladder in DNA retardation assay when OmpR and PrS₂-OmpR are mixed in different ratios.

Recently, we carried out further characterization of the detailed molecular mechanism of OmpR binding to DNA. During the course of these experiments, we discovered that (a) PrS₂-OmpR is expressed at a significantly higher level in pET expression system than wild-type OmpR, and (b) OmpR solubility in solution was dramatically (more than 20 fold) improved by protein S fusion.

### Significance

1. The proposed protein S-tag (PST) vector using pCold in E. coli is a most versatile expression system allowing (a) very high level of expression, (b) expression of proteins which cannot be easily expressed by any other expression systems, (c) expression of unstable proteins, and (d) dramatic enhancement of solubility of proteins, which otherwise would be expressed in inclusion bodies. These features provide tremendous advantage in labeling target proteins with isotopes (¹⁵N and ¹³C) for NMR study.
2. The proposed PST vector system in mammalian cells allows identification of the formation of protein complexes and protein-protein interaction. In particular, the system may enable one to identify protein factors, which form complexes only in living cells.
3. The use of myxospores is notable in following three respects; (a) it is very easy to prepare M. xanthus cultures, and the final purified myxospores are relatively inexpensive, (b) they are uniform and can be converted into completely biologically inactive particles by irradiation (in order to prevent germination) so that they can be reused for an indefinite number of experiments, (c) the density of myxospores is relatively low making it easier to resuspend into a homogeneous suspension.
4. We found that protein S can still bind to myxospores even in the presence of 4 M urea. Therefore, although some proteins fused to protein S form inclusion bodies, it is possible to refold these proteins after solubilizing with 4 M urea and purify using myxospores. In fact, a similar approach is successfully used for His-tagged proteins.
5. The proposed PST technology of the invention is novel and highly versatile, and is expected to make a major contribution to protein technology.

### C. Preliminary Studies

### 1. PrS₂ Tag Fused to OmpR Does Not Affect the Properties of OmpR

OmpR, a transcription factor of E. coli, was fused to two tandem repeats of the N-terminal domain (residues 1-92) of protein S (termed PrS₂-OmpR) to determine the exact number of OmpR-binding sites on the upstream region of ompF promoter (Harlocker, S. L., et al. 1995. J Biol Chem 270:26849-56). OmpR is a response regulator involved in a two-component signal transduction system and its cognate histidine kinase is EnvZ. EnvZ phosphorylates OmpR at the conserved Asp residue (D55) to generate phosphorylated OmpR (OmpR-P) and can also dephosphorylate OmpR-P. We examined if PrS₂ tag fused to OmpR affects the properties of OmpR by monitoring (i) the complex formation between the cytoplasmic domain of EnvZ (EnvZc) and OmpR, (ii) the phosphorylation of OmpR and dephosphorylation of OmpR-P, and (iii) DNA binding of OmpR.

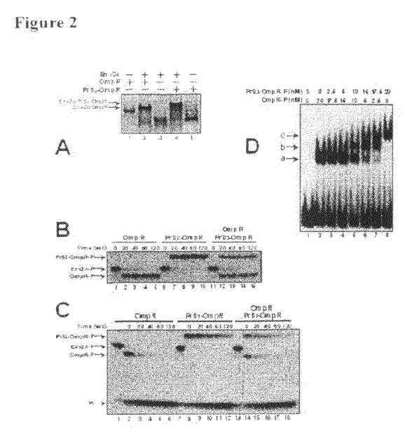

It is known that the EnvZc/OmpR complex can be easily detected by native-PAGE (Yoshida, T., et al. 2002. Mol Microbiol 46:1273-82). As shown in FIG. 2A, when purified OmpR and EnvZc were mixed and subjected to 10% native-PAGE, they formed a complex (lane 2), which migrates slower than OmpR and EnvZc (lanes 1 and 3, respectively). We tested if PrS₂ tag affects the complex formation between EnvZc and OmpR. When purified EnvZc and PrS₂-OmpR were mixed, these, too, form a complex (lane 4), demonstrating that PrS₂ tag fused to OmpR does not interfere with the complex formation between EnvZc and OmpR.

Next, we examined if enzymatic properties of EnvZc and OmpR are affected by fusion of OmpR with PrS₂ tag. First, phosphorylation of PrS₂-OmpR by EnvZc-P was tested. When OmpR was mixed with purified ³²P-labeled phosphorylated EnvZc (EnvZc-P), the phosphoryl group on EnvZ was instantly transferred to OmpR and this reaction was completed in 120 sec (lanes 2-5, FIG. 2B). When this reaction was carried out with PrS₂-OmpR, EnvZc-P was able to phosphorylate PrS₂-OmpR and the result was almost identical to the non-tagged OmpR (lanes 7-10, FIG. 2B). The competition experiment further proved that OmpR and PrS₂-OmpR were equally good substrates for EnvZc-P (lanes 12-15, FIG. 2B). Secondly, dephosphorylation of phosphorylated PrS₂-OmpR was examined. After generating OmpR-P as described above, dephosphorylation of OmpR-P was observed upon the addition of ADP, which is known as a co-factor for this reaction and stimulates the phosphatase activity of EnvZc. OmpR-P was quickly dephosphorylated and Pi was released as shown in lanes 3-6, FIG. 2C. Although in case of PrS₂-OmpR, this reaction was slowed by PrS₂ tag, phosphorylated PTS₂-OmpR was dephosphorylated by EnvZc (lanes 9-12). The competition experiment between OmpR and PrS₂-OmpR again demonstrates that OmpR-P and PrS₂-OmpR serve equally as a substrate for EnvZc (lanes 15-18).

Finally, we tested if binding of PrS₂-OmpR to DNA is affected by PrS₂ tag (FIG. 2D). It is known that two OmpR-P molecules can bind to each 20-bp binding site to form a dimer-like structure on DNA. When OmpR-P was mixed with 30-bp DNA fragment (F1F2a), which has the 20-bp F1 site and a half site of F2 (F2a), the OmpR-P/F1F2a complex was detected [band (a), lane 2]. When PrS₂-OmpR was mixed, the PrS₂-OmpR/F1F2a complex migrated much slower than the OmpR-P/F1F2a complex and the migration of the PrS₂-OmpR/F1F2a complex band was further retarded [band (c), lane 8]. Once various mixtures of OmpR and PrS₂-OmpR were mixed with F1F2a, another new band appeared between band (a) and band (c) [band (b) in lanes 3-7]. This band resulted from the binding of one OmpR molecule and one PrS₂-OmpR molecule to F1F2a, indicating that PrS₂ tag does not affect the DNA binding ability of OmpR and also the interaction between the OmpR molecules bound on DNA.

These data clearly demonstrate that PrS₂ tag fused to OmpR does not change the properties of OmpR, suggesting that the PrS₂ tag domain is folded independently of OmpR.

### 2. Binding of PrS₂-OmpR to Myxospores is Ca⁺⁺-Dependent

In order to examine if newly constructed PrS₂ fragment (two tandem repeats of N-terminal domain of protein S), as well as PrS₂-OmpR are able to bind to myxospores, we prepared myxospores from 10-days mature fruiting bodies of M. xanthus. We were able to harvest approximately 2 g of purified myxospores from a 1.5-L culture. Protein S (Inouye, M., et al., 1979. Proc Natl Acad Sci USA 76:209-13) and protein C (McCleary, W. R., et al. 1991. J Bacteriol 173:2141-5) were completely removed from myxospores by washing these with 20 mM EDTA before use.

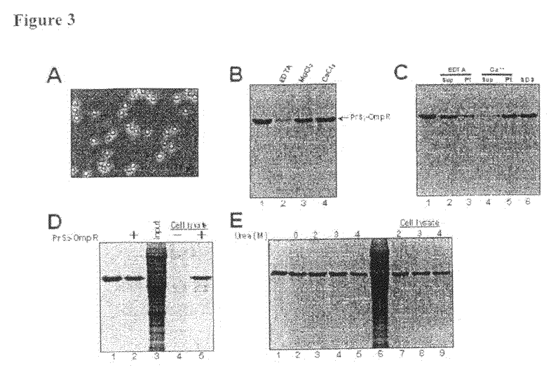

First, we tested if PrS₂-OmpR binds to the protein S-less myxospores in a Ca⁺⁺-ion dependent manner. Purified PrS₂-OmpR (3 μg) was mixed with myxospores in 100 μl of 50 mM Tris-HCl (pH 8.0) buffer containing 50 mM KCl in the presence of 20 mM EDTA, 10 mM MgCl₂, or 10 mM CaCl₂, and the final mixtures were incubated at 4° C. for 1 hr. Myxospores were collected by centrifugation at 6,500 rpm for 3 min using a microcentrifuge (Biofuge Pico, SORVALL) and the myxospore pellets were washed once with the respective binding buffer. The final myxospore pellets were suspended in 20 μl of SDS-loading buffer [20 mM Tris-HCl (pH 6.8), 40 mM β-mercaptoethanol, 0.8% (w/v) SDS, 4% glycerol, and 0.04% (w/v) bromophenol blue]. The myxospore suspensions were incubated in a boiling water bath for 5 min. By this treatment only proteins bound to the myxospore surface are solubilized, but the intracellular proteins of myxospores arc not affected. These samples were then analyzed by 17.5% SDS-PAGE. As shown in FIG. 3B, PrS₂-OmpR is bound to myxospores in the presence of MgCl₂ (lane 3) and CaCl₂ (lane 4) but not in the presence of EDTA (lane 2).

Since protein S bound to myxospores can be released by the addition of EDTA, we also examined if EDTA can release PrS₂-tagged protein bound to myxospores. After PrS₂-OmpR was incubated with myxospores for 1 hr at 4° C., the myxospore pellet was incubated in EDTA buffer [50 mM Tris-HCl (pH 8.0), 50 mM KCl, and 20 mM EDTA] for 15 min at room temperature. PrS₂-OmpR was released into the supernatant in the presence of EDTA (lane 2, FIG. 3C), while PrS₂-OmpR was still bound to myxospores in the presence of Ca⁺⁺ (lane 4, FIG. 3C). These results demonstrate that the PrS₂ fragment in PrS₂-tagged OmpR fully retains the ability to bind to the surface of myxospores in a Ca⁺⁺-dependent manner. Note that although in the presence of Mg⁺⁺ ion protein S can bind to myxospores, its binding is known to be weaker than in the presence of Ca⁺⁺(Inouye, M., et al., 1979. Proc Natl Acad Sci USA 76:209-13). Therefore, for all the experiments with PrS₂-tagged proteins, Ca⁺⁺ ion is included in the binding buffer.

Our next question was how specifically a PrS₂-tagged protein can bind to myxospores. To achieve one-step affinity purification of a protein, it is very important to minimize non-specific binding of contaminating proteins to myxospores. PrS₂-OmpR binding to myxospores was tested in the presence of E. coli BL21(DE3) cell lysate (lane 3, FIG. 3D). Under this condition, PrS₂-OmpR specifically binds to myxospores and only minor contaminants were observed by Coommassie Blue staining (lane 5, FIG. 3D). Interestingly, the PrS₂-OmpR binding to myxospores was not affected even in the presence of urea up to 4 M (FIG. 5E).

### Application of PST Technology

One of advantages in affinity protein purification is that binding and eluting of an affinity-tagged protein can be carried out under physiological conditions, which allows one to study protein-DNA or protein-protein interactions. We tested if the PST (Protein S Tag) technology allows one to detect protein-DNA interactions using PrS₂-OmpR.

### Isolation of the PrS₂-OmpR/DNA Complex

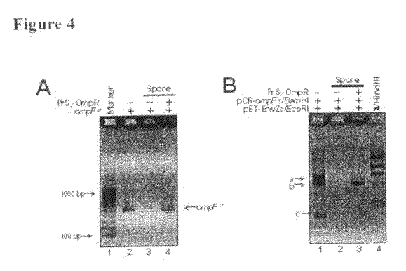

Since OmpR can bind to DNA as shown in FIG. 2D, we tested if the PrS₂-OmpR/DNA complex can be isolated using myxospores. For this purpose, a 500-bp DNA fragment containing the sequence from the upstream region of the ompF promoter (ompF^{P}) was used. First, ompF^{P} DNA and PrS₂-OmpR (3 μg) was mixed and the mixture was incubated for 30 min at room temperature. Myxospores were then added to the mixture. After 1-hr incubation at 4° C., myxospores were collected by centrifugation. The final pellets were suspended in DNA loading buffer containing urea [20 mM Tris-HCl (pH 7.5), 5 M urea, 40 mM β-mercaptoethanol, 0.8% (w/v) SDS, 4% glycerol, and 0.04% (w/v) bromophenol blue] and then the supernatants were analyzed for DNA binding by agarose gel electrophoresis. As shown in FIG. 4A, ompF^{P} DNA was detected in the sample in which the DNA fragment was mixed with myxospores in the presence of PrS₂-OmpR (lane 4), while it was not detected in the reaction carried out in the absence of PrS₂-OmpR (lane 3).

Next, a linearized plasmid DNA containing ompF^{P} fragment (pCR-ompF^{P}, 4.5 kbp) was prepared by digesting pCR-ompF^{P} with BamH I [band (b)] and pET-EnvZc was also digested with EcoR I to release EnvZc fragment (1.5 kbp) [band (c)]. Note that pET-EnvZc was constructed by inserting the EnvZc fragment at the EcoR I site of pET11a vector. Therefore, EcoR I digestion of pET-EnvZc yields two bands (a), pET11a vector, and (c), EnvZc fragment. Using the DNA mixture of pCR-ompF^{P} digested by BamH I, and pET-EnvZc digested by EcoR I, the same experiment as described in FIG. 4A was carried out in the absence or presence of PrS₂-OmpR. As shown in FIG. 4B, in the presence of PrS₂-OmpR only one band corresponding to the pCR-ompF^{P} fragment was detected from the myxospore pellets (lane 3) while in the absence of PrS₂-OmpR, DNA was not detected (lane 2). These results demonstrate that myxospores are able to trap both PrS₂-OmpR/0.5-kbp ompF^{P} DNA complex and PrS₂-OmpR/4.5-kbp pCR-ompF^{P} DNA fragment complex. Notably, the procedures used here were gentle enough to be used for isolation of intact protein-DNA complexes.

### Research Design and Methods

On the basis of the preliminary results described above, we can pursue the following two studies. PST technology can enable us to solve the structures of proteins, which cannot be determined by the conventional X-ray and NMR technologies.

Study #1—Construction of Protein S Tag (PST) Expression System in E. coli and Mammalian Cells and the Use of Myxospores for Affinity Purification

In this experiment, we can develop two PrS₂-OmpR-tagged protein expression systems; (1) E. coli PST-expression system: this can be constructed using a high expression cold-shock vector, pCold, recently developed in our laboratory (Qing, G., et al. 2004. Nat Biotechnol 22:877-82). The final product is termed, pCold(PST), in which a protein of interest is fused to PrS₂ tag at the N-terminal end and the expression of fusion protein is induced at a very high level upon cold shock treatment. (2) Mammalian PST-expression system: this can be constructed by using a tetracycline-inducible vector. This system is designed to isolate a multiple protein complex formed in living cells. The complex associating with a PrS₂-tagged protein can be trapped using myxospores and proteins in the complex will be identified by mass spectroscopy. In the third approach of this Aim, we can establish a method for preparation of highly purified myxospores devoid of protein S and protein C for the use of affinity protein purification of PrS₂-tagged proteins and protein complexes.

Approach #1: PST Expression System in E. coli

Cold-shock vectors (pCold vectors) were recently developed in our laboratory and have proved to be complementary to the pET vector system (Qing, G., et al. 2004. Nat Biotechnol 22:877-82). The expression of a protein of interest is induced at a very high level upon cold shock (15° C.). A protein of interest under the cold-shock expression system is produced with a low background production of cellular proteins. Thus the protein can be almost exclusively labeled with ¹⁵N and ¹³C isotopes when the cell is grown in media containing ¹⁵NH₄Cl and ¹³C-glucose, which enables one to perform NMR structural studies of the protein simply using cell lysate without further purification (Qing, G., et al. 2004. Nat Biotechnol 22:877-82). We can adapt the pCold vector system to construct the PST system in E. coli.

a. Construction of pCold(PST)

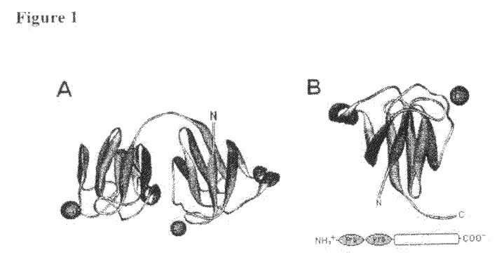

The pColdIII vector (Qing, G., et al. 2004. Nat Biotechnol 22:877-82); see FIG. 1) consists of the cspA promoter (cspA, the major cold-shock gene in E. coli), the cspA 5′-UTR (159 bases), the initiation codon, the translation enhancing element (TEE) and the multi-cloning site. Using this plasmid the DNA fragment for PrS₂ (184 residues) can be inserted between TEE and the multiple cloning site. After the multiple cloning site a His₆ tag can be inserted, with a thrombin cleavage site (Leu-Val-Pro-Arg↓Gly-Ser) at the N-terminal end of the His₆ tag so that the His₆ tag can easily be removed from the protein. This newly constructed vector is termed pCold(PST) (FIG. 5). Notably, the His₆ tag could also be added at the C-terminal end of PrS₂-tagged protein, in which case a DNA fragment for a protein of interest can be inserted in frame with the His₆ tag. The DNA fragment can also be prepared by PCR to have a termination codon to eliminate His₆ tag at the C-terminal end.

For the NMR structural study of a protein described in Experiment #2, the linker between protein S and the protein of interest is essential to make the protein freely move freely even if the N-terminal PrS₂ tag is firmly fixed on the myxospore surface (see Experiment #2). Although the C-terminal end of the protein S fragment (residues 1 to 92) has a random structure consisting of six amino acid residues (—⁸⁷VPVQPR⁹²—), we can optimize the length of the linker if necessary (see Specific Aim #2 for detail). TEV cleavage site (Glu-Asn-Leu-Tyr-Phe-Gln↓Gly) is added after the C-terminal end of PrS₂ so that the cloned gene product can be detached from PrS₂ by TEV treatment. The final vector is termed pCold(PST).

b. Application of pCold(PST) to Model Proteins

We can use pCold(PST) to express three model proteins; CspA, EnvZ domain A and EnvZ domain B. CspA is a small β-barrel protein consisting of 70 residues and functions as an RNA chaperone (Jiang, W., 1997. J Biol Chem 272:196-202); and its structure has already been determined by NMR (Newkirk, K., et al. 1994. Proc Natl Acad Sci USA 91:5114-8). EnvZ domain A (67 residues) structure, determined by NMR, consists of two helices forming a hair-pin structure (Tomomori, C, et al. 1999. Nat Struct Biol 6:729-34). This fragment is the central dimerization domain having the His residue as the autophosphorylation site for EnvZ. EnvZ is a histidine kinase involved in osmoregulation of ompF and ompC genes in E. coli (see Egger, L. A., et al. 1985. Genes Cells 2:167-84, Forst, S. A., et. al. 1994. Res Microbiol 145:363-373 for review). EnvZ domain B is a α/β protein (161 residues) and serves as the ATP-binding domain for EnvZ. Its three-dimensional structure has been determined by NMR (Tanaka, T., et al. 1998. Nature 396:88-92).

Since all of these protein structures (β, α, and α/β) have been determined by NMR, they are ideal model proteins for Specific Aim #2. Upon expression of these proteins using pCold(PST), they can be purified with the use of Ni-NTA resin. Their individual biochemical properties, such as RNA binding for CspA, phosphorylation by EnvZ for EnvZ domain A and ATP binding for EnvZ domain B, will be examined in comparison with their non-tagged proteins. On the basis of the results obtained with PrS₂-OmpR (see Preliminary Results section), all these proteins are expected to retain their biochemical properties. Any changes observed in the biochemical properties, would be due to the His₆ tag at the C-terminal end. In this case, we can remove the C-terminal His₆ tag by thrombin, and reexamine their biochemical properties. All of these proteins are expected to be produced at very high levels (30-50% of total cellular protein) by cold-shock treatment [at 15° C. for 12 hr; (Qing, G., et al. 2004. Nat Biotechnol 22:877-82)]. For Specific Aim #2, we can label these proteins with ¹⁵N using the method established in our laboratory (Qing, G., et al. 2004. Nat Biotechnol 22:877-82).

c. Enhancement of Protein Expression and Solubility

Since the expression and solubility of OmpR was dramatically enhanced by its fusion with PrS₂ tag, we can use this PrS₂ tag as a general protein expression system. We can examine the effectiveness of pCold(PST) for the expression of set of genes used as model genes. This set is called “core 50”, and contains genes from Arabidopsis thaliana, Caenorhabditis elegans, Drosophila melanogaster, and Homo sapiens and has been used to test various different expression systems such as pET, pCold and wheat germ system (http://www-nmr.cabm.rutgers.edu/bioinformatics/ZebaView/). Therefore, the use of “core 50” is ideal to test pCold(PST). We can test genes, particularly those whose products are poorly expressed or produced in inclusion bodies, for their expression and solubility using pCold(PST) (see Qing, G., et al. 2004. Nat Biotechnol 22:877-82) for methods).

### Approach #2: PST Expression System in Mammalian Cell

As we have shown above, a protein-DNA complex can be isolated using PrS₂-tagged OmpR and myxospores (FIG. 4), this method can also be applied for the isolation of other protein complexes and for the identification of protein-protein interactions. The PST technology has a great potential in mammalian systems, in particular, for the isolation of a multi-protein complexes that is only formed in living cells. Therefore, we can construct a tetracycline-inducible PrS₂-fusion expression system, and test the system with TATA-binding protein (TBP) as a model system. This is known to form a multiprotein complex that plays an important role in eukaryotic transcription initiation.

a. Construction of Tetracycline-Inducible PST System: T-REx(PST)

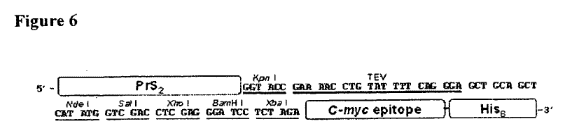

To construct a tetracycline-inducible PST system, we can use a plasmid vector, pcDNA4/TO/myc-His (Invitrogen), which is tetracycline-inducible vector and has the C-terminal myc epitope and His₆ tag. PrS₂ fragment can be inserted by using Hind III and Kpn I sites in the multiple cloning site of this vector. TEV cleavage site and a new multiple cloning site can also be inserted by using Kpn I and Xba I sites in the multiple cloning site as shown in FIG. 6, so that PrS₂ tag fused to a protein of interest can be removed if necessary. Since this PrS₂-tagged protein has a c-myc epitope at the C-terminal end, it can be detected by simple Western blotting.

In order to establish a stable cell line that expresses PrS₂-tagged protein, we can use T-REx 293 cell line as a host cell (Invitrogen). This T-REx 293 is a Human Embryonic Kidney 293 cell line that constitutively expresses the Tet repressor, which can repress transcription of a PrS₂-tagged protein until an inducer, tetracycline, is added. T-REx(PST) harboring PrS₂-tagged protein can be transfected into T-REx 293 cells using PolyFect transfection reagent (QIAGEN), and stable transformants will be selected for 2 weeks on Dulbecco's modified Eagle medium (DMEM) supplemented with 10% bovine calf serum and drugs, 5 μg/ml blasticidin and 40 μg/ml Zeocin. Drug-resistant clones will be analyzed for expression of PrS₂-tagged protein after inducing with tetracycline, and the clone, which expresses the PrS₂-tagged protein at the highest level, will be used for this study.

b. Application of T-REx(PST) System to a Model Protein

We can test the PST system in mammalian cells using human TATA-binding protein (TBP) as a model protein. Involvement of TBP in transcription initiation is well characterized and it is a core protein that forms a multiprotein-DNA complex (Hori, R., and M. Carey, et al. 1994. Curr Opin Genet Dev 4:236-44, Maldonado, E., et al. 1995. Curr Opin Cell Biol 7:332-61, Roeder, R. G., et al. 1991. Trends Biochem Sci 16:402-8). This multiprotein-DNA complex formation is initiated through binding of TBP to a TATA element and its association with transcription factor IIB (TFIIB), one of general transcription factors, to finally assemble a preinitiation complex (PIC) containing RNA polymerase II and other known general transcription factors (Zawel, L., et al. 1993. Prog Nucleic Acid Res Mol Biol 44:67-108).

First, we can prepare a stable cell line as described above and can harvest the induced cells and also the non-induced cells as a control. We can examine if PrS₂-TBP can be isolated from cell lysate by mixing with myxospores. After confirming that PrS₂-TBP can be isolated by myxospores, we can compare proteins that are bound to myxospores from induced and non-induced cells. If protein bands detected in the PrS₂-TBP induced cells are different from those in non-induced cells, we can identify these proteins by either Western blot or Mass spectrometric fingerprinting, which can be carried out on a MALDI-TOF mass spectrometry at our medical school core facility by Takeshi Yoshida who uses it routinely for our laboratory.

If using PrS₂-TBP and myxospores associating factors are successfully isolated, we can apply this system to study protein(s) interacting with Su(z)12, suppressor of zeste-12, involved in histone H3-H1 methyltransferase activity (Kuzmichev, A., et al. 2002. Genes Dev 16:2893-905).

### Approach #3: Preparation of Highly Purified Protein S-Depleted Myxospores

For affinity purification of PrS₂-tagged proteins and isolation of protein complexes it is essential to obtain highly purified myxospores. In this approach, we can establish the methods to prepare a large quantity of purified myxospores that are depleted with protein S and protein C assembled on the surface. We can also establish a method to inactivate myxospores so that they do not to germinate under any condition, and can thus be used as a reusable biomaterial. Since myxospores are biologically prepared, the cost for preparation of purified myxospores is highly economical compared with other affinity resins such as Ni-NTA, streptavidin, glutathione, amylose, and calmodulin resin. Furthermore, M. xanthus and its spores are not pathogenic and completely safe to handle.

a. Preparation of Myxospores

Myxococcus xanthus FB(DZF1) is used a source of myxospores. For growing cells, Casitone-yeast extract (CYE) medium [1% casitone, 0.5% yeast extract, 10 mM Tris-HCl (pH 7.6), and 8 mM MgSO₄] are used and for fruiting body formation, clone fruiting (CF) agar [0.015% casitone, 0.2% Na-citrate, 0.02% (NH₄)₂SO₄, 10 mM Tris-HCl (pH 7.6), 8 mM MgSO₄, 0.1% Na-pyruvate, 1 mM potassium phosphate buffer (pH 7.6), and 1.5% Bacto agar] plates are used. Before use the plates are dried at 30° C. over night and then at room temperature for 5 days.

The cells are grown at 30° C. until they reach exponential phase (100 Klett units). Cells are harvested by centrifuging at 4000×g for 10 min at room temperature. The cell pellet is washed with equal volume of TM buffer [10 mM Tris-HCl (pH 7.6) and 8 mM Mg₂SO₄]. The cell pellet is resuspended in TM buffer to achieve the concentration of 4000 Klett units/ml. This concentrated cell suspension is spotted on CF agar plates (4 μl per spot, 144 spots per plate), and the plates are incubated at 30° C. for 10 days. Fruiting bodies are harvested by gentle scraping of the surface of the plate and are suspended in cold TM buffer. The cells are sonicated three times for 5 min to break vegetative cells. The myxospores are washed several times with TM buffer and finally are resuspended in 10 mM Tris-HCl (pH 7.6) buffer. The myxospores are kept at 4° C. until use.

Purified myxospores are incubated in 10 mM Tris-HCl (pH 8.0) buffer containing 25 mM EDTA at room temperature to remove native protein S (Inouye, M., et al., 1979. Proc Natl Acad Sci USA 76:209-13) and protein C (McCleary, W. R., et al. 1991. J Bacteriol 173:2141-5) from their surface for 1 hr and after several washes this step is repeated one more time. This EDTA-treated myxospores are then heated at 60° C. for 10 min to inactivate proteases if any. As shown in FIG. 3A, these treated myxospores have uniform spherical shape and are used in our study.

b. Maintenance of Well-Suspended Myxospores

Purified myxospores are uniform in size (diameter of 1 μm; see FIG. 3A), and slightly denser than water (thus if a myxospore suspension is kept for long periods of time they sediment at the bottom of a tube in a buffer solutions). In order to maintain a well-suspended state of myxospores, we tested the effect of sucrose on myxospore suspension. Myxospores were suspended in 0.3 ml of 0, 5, 10, 15, and 20% sucrose solution at room temperature. They were well suspended without sedimenting at the lowest concentration of 5% sucrose (data not shown). We can use this condition for experiments designed for NMR structural study.

c. Inactivation of Myxospores

Myxospores germinate under a certain condition such as in the presence of 1 mM Ca⁺⁺ ion and casitone medium (Otani, M., et al. 1995. J Bacteriol 177:4261-5). For the PST technology, it is essential to prevent myxospore germination so that they can be reused many times. We can test the following methods to completely inactivate the ability of myxospore to germinate.

Effect of NaN₃—To test if NaN₃ (1 mM) prevents germination of myxospores, we can incubate myxospores in a germination medium (0.2% casitone containing 1 mM Ca⁺⁺) with and without NaN₃ at 30° C. for 10 hours. Inhibition of germination can be monitored by monitoring the refractility of myxospores under a phase microscope.

Effect of Heat Treatment—To test if treating the myxospores with high temperature prevents their germination, we can incubate them at 70, 80, 90, and 100° C. for 30 min. We can determine the optimum heat treatment by which the myxospores lose their ability to germinate, but retain their protein S binding property.

Effect of γ-Ray Irradiation—If both methods described above are ineffective in inhibiting germination of myxospores, we can use γ-ray irradiation to kill myxospores. Note that myxospores are resistant to UV irradiation.

### Study 2 Nuclear Magnetic Resonance (NMR) Structural Study of Protein S-tagged Proteins Trapped on Myxospores

If a protein consists of two completely independent domains, X and Y, linked through a flexible linker, and there is no interaction between the two domains, we hypothesize that it is possible to eliminate NMR signals from domain X by restricting the isotropic movement of domain X. This may be achieved by domain X firmly binding to the surface of a particle, which is well suspended in solution. Under this condition domain Y still retains its isotropic movement in solution as it is connected to domain X through a highly flexible linker. Therefore, every peak of the HSQC spectrum of domain X may be broadened while every peak of the HSQC spectrum of domain Y remains sharp for structural analysis. If successful, the PST technology proposed thus will open a novel exciting avenue for the following studies:

1. NMR Structural Study of Proteins that are Poorly Soluble.

For NMR structural study a high concentration of a protein of interest is required to get a high signal-to-noise ratio. It is absolutely essential that the protein is completely soluble without any aggregates or precipitates. If a protein of interest is not stable at high concentrations, it is expected that this problem will be overcome by using the PST technology. In addition to the fact that PrS₂-tagging enhances protein solubility, the binding of PrS₂-tagged protein to myxospores may avoid aggregation of the protein. Furthermore, the concentration of PrS₂-tagged protein can be concentrated much more than the untagged protein.

2. NMR Structural Study of Proteins that are Expressed Only When They are Fused to PrS₂ Tag.

### 3. The Structural Study of Membrane Proteins.

Membrane proteins are not usually expressed at high levels. Using the proposed PST technology, one may be able to achieve one-step purification of the membrane proteins dissolved in an appropriate detergent. The myxospore-bound membrane proteins can thus be prepared at very high concentrations needed for their NMR structural studies.

### Experimental Approaches

We can test the above hypothesis by using myxospores and ¹⁵N-lableled PrS₂-tagged proteins from Example 1. In the above hypothesis, PrS₂ tag serves as domain X and the proteins fused to the PrS₂ tag as domain Y. In order to achieve our goal, it is important to establish the method for the preparation of large quantities of reusable myxospores in a cost effective manner as proposed in Example 1.

### Approach #1: Effect of Binding of Myxospore on the HSQC Spectrum of Protein S

The PrS₂ fragment (184 residues) can be expressed at a very high level with a pCold vector, thus 20 mg purified ¹⁵N-labeled PrS₂ fragment can be easily obtained from a 500-ml culture. Using ¹⁵N-labeled PrS₂ fragment, we can compare its heteronuclear single quantum coherence (HSQC) spectra in the presence and absence of myxospores. It is expected that upon the addition of myxospores in the presence of 10 mM CaCl₂, every peak of the HSQC spectrum of PrS₂ will become broadened, because the molecular motion of PrS₂ will be greatly restricted by binding to myxospores. We can make sure that this effect of the addition of myxospores is reversible by adding 10 mM EDTA. Since EDTA releases bound PrS₂ from myxospores, every peak of the HSQC spectrum should become sharp again.

### Approach #2: HSQC Spectra of PrS₂-Tagged Model Proteins

Upon confirming that myxopores cause broadening of the peaks of HSQC spectra of PrS₂, we can then take HSQC spectra of ¹⁵N-labeled PrS₂-tagged CspA, EnvZ domain A, and EnvZ domain B from Specific Aim #1 in the presence and absence of myxospores. These spectra can then be analyzed using the following criteria:

Those peaks broadened in all three spectra as a result of the addition of myxospores are expected to be from PrS₂ tag. These broadened spectra are expected to be very similar to that of PrS₂ in the presence of myxospores. If this is the case, our hypothesis will be proven to be correct. Therefore all these peaks may be removed from the HSQC spectra obtained in the presence of myxospores.

The HSQC spectra thus obtained (after the remove of all peaks from PrS₂ tag) can be compared with the HSQC spectra of the individual proteins, all of which are available in our laboratory, as these NMR structures have been determined by us in collaboration with Dr. G. Montelione [for CspA: (Newkirk, K., et al. 1994. Proc Natl Acad Sci USA 91:5114-8)] and Dr. Ikura [for EnvZ domain B and A; (Tanaka, T., et al. 1998. Nature 396:88-92. Tomomori, C., et al. 1999. Nat Struct Biol 6:729-34)].

At present, the component(s) on the myxospore surface that triggers the autoassembly of protein S, is not known. It remains to be examined how the surface density of PrS₂-tagged protein affect the HSQC spectra of a protein of interest. If the surface density of a PrS₂-tagged protein is too high, there may be intermolecular interaction between proteins, which can restrict the free movement of proteins, resulting in peak broadening. This can be examined by using different amounts of myxospores with the same amount of a PrS₂-tagged protein for NMR analysis. The addition of more myxospores will reduce the density the PrS₂-tagged protein bound per myxospore and as a result the broadening effect may be avoided. Another important factor, which may influence the quality of HSQC spectra, is the length of the flexible linker. We can examine the effect of the length of the linker by adding the (GGGGS)ₙ (n=1, 2, 3 . . . ) residues one at a time. Through these experiments the optimal linker for the PST technology can be established.

If these approaches are successful, we can apply the PST technology to some of proteins tested in Approach #1 of Specific Aim #1 in order to test the general applicability of the technology. In particular, we can test (a) those proteins that are expressed well only when they are tagged with PrS₂, (b) those, which are expressed only at low levels. In these cases, their binding to myxospores may substantially increase their concentration for NMR study and (c) those proteins, which are soluble only in 4 M urea. As PrS₂ is fully capable of binding to myxospores in 4 M urea, the PrS₂-tagged proteins are easily purified in the presence of 4 M urea (see Preliminary Results section). Note that as the solution used for myxospore suspension can be easily replaced with simple centrifugation, 4 M urea solution can be readily replaced with a buffer solution without urea. This ease of buffer replacement can also provide a method for quick screening for optimal buffer conditions for NMR experiments.

### Obtaining Optimal Results

How similar the spectra of PrS₂-tagged proteins are to the spectra of non-tagged proteins is a key question for determining the feasibility of the proposed PST technology for NMR structural study of proteins. Although the binding of PrS₂-tagged proteins to myxospores may unpredictably affect resolution (broadening effect) of each peak of the PrS₂ HSQC spectrum, the N-terminal Ca⁺⁺-binding domain consists of a very stable globular structure mainly composed of β-strands (Bagby, S., et al. 1994. Proc Natl Acad Sci USA 91:4308-12), and each Ca⁺⁺-binding unit of PrS₂ is resistant to tryptic digestion (Inouye, S., et al. 1981. J Bacteriol 148:678-83) and heat treatment (Bagby, S., et al. 1998. J Mol Biol 276:669-81). Therefore, when the 92-residue N-terminal domain binds to myxospores through its Ca⁺⁺-binding site, it is likely that the entire domain is rigidly fixed on the myxospore surface. This will cause broadening of every peak of the PrS₂ HSQC spectrum. However, it is also possible that the quality of HSQC spectrum of the domain (domain Y) fused to PrS₂ may be affected if the movement of domain Y is restricted by PrS₂ tag.

For this reason, the linker connecting PrS₂ tag and domain Y is considered to play an important role in free movement of domain Y. It has to be determined how the length of the linker affects the quality of the HSQC spectrum of domain Y. We can first test this question by adding the (GGGGS)ₙ linker (n=1, 2, 3 - - - ) using PrS₂-CspA as a model system. The addition of the four-residue sequence one at a time is expected to add more freedom to the movement of domain Y, which in turn should sharpen the peaks for CspA HSQC spectrum. By comparing the HSQC spectrum with that of purified CspA, we should be able, to optimize the length of the flexible linker. Upon establishing the optimal linker length, we will confirm if this optimal length is also applicable to other model systems as well.

While the present invention has been described with reference to the specific embodiments thereof, it should be understood by those skilled in the art that various changes may be made and equivalents may be substituted without departing from the true spirit and scope of the Invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A vector containing a multiple cloning site comprising a nucleic acid sequence encoding a PrS tag or a PrS₂ tag from Myxococcus xanthus.

2. The vector of claim 1, wherein the multiple cloning site comprises SEQ ID NO: 1.

3. The vector of claim 2, wherein the multiple cloning site comprises SEQ ID NO: 2.

4. The vector of claim 1, wherein the vector is a high expression cold shock vector.

5. The vector of claim 4, wherein the cold shock vector is a pColdIII vector.

6. The vector of claim 4 expressed in Escherichia coli.

7. The vector of claim 1, wherein the vector is a tetracycline-inducible PST expression system.

8. The vector of claim 7 expressed in a mammalian host cell.

9. The vector of claim 8, wherein the mammalian host cell is from a TREx 293 human embryonic kidney cell line.

10. A method for enhancing solubility of a target protein, comprising:
a) fusing a nucleic acid sequence encoding at least one N-terminal domain of Protein S (PrS tag) from Myxococcus xanthus to a nucleic acid sequence encoding the target protein to create a nucleic acid sequence encoding a PrS tagged target protein;
b) positioning the Protein S tagged target protein of step (a) into a vector;
c) transforming a host cell with the vector; and
d) culturing the host cell under conditions suitable for gene expression,
whereby the expressed PrS tagged target protein is more soluble than the untagged target protein.

11. The method of claim 10, wherein the nucleic acid sequence encodes a PrS₂ tag having two tandemly repeated N-terminal domains of Protein S.

12. The method of claim 10, wherein after performing step (d), the PrS tag is optionally cleaved from the target protein using a protease.

13. The method of claim 10, wherein the host cell is a prokaryote.

14. The method of claim 1, wherein the target protein is from a prokaryote or a eukaryote.

15. The method of claim 1, wherein the vector is an Escherichia coli pCold expression system containing a multiple cloning site comprising the nucleic acid encoding the PrS tagged target protein.

16. The method of claim 1, wherein the host cell is a eukaryote.

17. The method of claim 1, wherein the vector is a mammalian tetracycline-inducible system.

18. The method of claim 1, wherein the N-terminal domain nucleic acid sequence encodes from 85-100 amino acid residues.

19. The method of claim 1, wherein the N-terminal domain nucleic acid sequence encodes 93 amino acid residues.

20. A PrS-tagged protein molecule bound to one or more myxospores of Myxococcus xanthus.

21. A method for purifying a PrS-tagged or PrS₂-tagged protein comprising contacting the tagged protein with an affinity resin comprising myxospores of Myxococcus xanthus, thereby immobilizing the PrS-tagged or PrS₂-tagged protein on the affinity resin.

22. The method of claim 21, wherein the PrS-tagged or PrS₂-tagged protein is contacted with the affinity resin in the presence of Ca²⁺ or Mg²⁺.

23. The method of claim 22, wherein the PrS-tagged or PrS₂-tagged protein is released from the affinity resin by adding an agent that chelates calcium.

24. The method of claim 23, wherein the agent that chelates calcium is EDTA or EGTA.

25. A protein purified according to the method of claim 21.

26. A protein of claim 25, wherein the protein is a PrS- or a PrS₂-tagged protein.

27. A kit comprising an affinity resin according to claim 21 and instructions for use in a method of purifying a PrS- or PrS₂-tagged protein.

28. A kit according to claim 27, further comprising components for producing a PrS- or PrS₂-tagged protein.

29. A method of claim 1, wherein the PrS- or PrS₂-tagged protein comprises a linker between the protein S and the target protein.

30. A method of claim 29, wherein the linker is a polypeptide.

31. A method of claim 30, wherein the polypeptide is from 3 to 10 amino acid residues.

32. A method of preparing a target protein for an analytical study, comprising preparing a PrS- or PrS₂-tagged target protein, and performing the analytical study.

33. The method of claim 32, wherein the analytical study is selected from the group consisting of nuclear magnetic resonance spectroscopy, drug screening assays, binding assays and X-ray crystallography or any study requiring a soluble target protein.

34. The method of claim 33, wherein the PrS- or the PrS₂-tagged target protein comprises a linker between the PrS tag or PrS₂-tag and the target protein.

35. The method of claim 34, wherein the linker is a polypeptide.

36. A method of claim 33, wherein the analytical study is nuclear magnetic resonance spectroscopy.

37. A method of characterizing a target protein comprising fusing at least one PrS tag or a PrS₂ tag to the target protein, performing nuclear magnetic resonance spectroscopy and analyzing data from the nuclear magnetic resonance spectroscopy, thereby characterizing the target protein.
